(19) United States
(12) Patent Application Publication
(10) Pub. No.: US 2005/0148839 A1
(43) Pub. Date: Jul. 7, 2005

(54) Method for non-invasive measurement of spinal deformity

(76) Inventors: Adi Shechtman, Nofit (IL); Gideon E. Strulesi, Sikat Beit Hakerem (IL); Dalia Dickman, Misgav (IL)

Correspondence Address: William H. Dippert;Reed Smith LLP, 599 Lexington Avenue, 29th Floor, New York, NY, 10022-7650, US

(21) Appl. No.: 10/732,164

(22) Filed: Dec. 10, 2003

(51) Int. Cl. A61B 5/05

(52) U.S. Cl. 600/407; 600/594

(57) ABSTRACT

An improved non-invasive method for measuring a spinal deformity of a patient whose spinal prominences representing the spinous processes are registered and their position mapped. The method comprises acquiring external physiological parameters indicative of position and orientation of the vertebrae; and calculating the deformity angle of the spine taking into account the registered spinal prominences and the external physiological parameters.

### FIELD OF THE INVENTION

The present invention relates to the measurement of spinal deformities, for example scoliosis. More particularly the present invention relates to a method of improved non-invasive assessment of spine deformity by incorporating physiological parameters indicative of position and orientation of the vertebra with mapping of the position of spinal prominences of a patient.

### BACKGROUND OF THE INVENTION

Scoliosis is a deformity of the spine that commonly affects children in their early and advanced phase of growth. Scoliosis is characterized by a 3 dimensional deformity of the spine that is composed of a spinal curvature, vertebral rotation and vertebral torsion. Observing an ordinary healthy spine, one can detect that it has natural curves. These curves round the shoulders and make the lower back curve slightly inward. But some people have spines that also curve from side to side. This condition of side-to-side spinal curvature is known as scoliosis. Scoliosis or spinal curvature may become noticeable from 9-16 years of age, but can also be noticed in some cases even much earlier. Hence, screening for scoliosis detection has been adopted in most of the U.S. schools and in most of the western world countries. Statistics indicates that up to 30% of the children that undergo a simple examination at school are advised to visit a pediatrician or orthopedist for a follow-up examination. 30% of these children will be identified as scoliotic patients and will undergo a more thorough set of examinations and require long-term treatment and follow-up. As the child grows, scoliosis begins to be a problem that can severely affect his life.

Basically the vertebral column is divided into five areas: cervical (7 vertebras), thoracic/dorsal (12 vertebrae), lumbar (5 vertebrae), sacral (5 vertebrae), coccygeal (4 vertebrae). When observing a healthy normal spine from the side view, one can detect that the spinal column (formed by the chained vertebrae) forms a 3 dimensional curve. In the upper trunk it normally has a gentle outward curve (Kyphosis) while the lower back has a reverse inward curve (Lordosis). Scoliosis is the abnormal curvature of the spine defined from the coronal view. It is typically a three-dimensional deformity of the spinal column and rib cage. It may develop in the following way:

As a single primary curve (resembling the letter C), or

As two curves (a primary curve along with a compensating secondary curve that forms an S shape).

Scoliosis most commonly develops in the region between the upper back (the thoracic area) and lower back (lumbar). This is referred to as the thoracolumbar area. It may also occur only in the upper back or lower back or in both. The physician attempts to define scoliosis by the following characteristics:
The shape of the curve.
Its location.
Its direction.
Its magnitude.
Its causes, if possible.

The degree of the curve is nearly always calculated using a technique known as the Cobb method. This examination is performed on an x-ray of the spine. In order to use the Cobb method, one must first decide which vertebrae are the end-vertebrae of the curve. These end-vertebrae are the vertebrae at the upper and lower limits of the curve, which tilt most severely toward the concavity of the curve. Theses vertebrae are marked 12 and 13 in FIG. 1. Once these vertebrae have been selected, two lines are then drawn, one along the upper endplate of the upper body and another along the lower endplate of the lower body. Perpendicular lines are then drawn from these lines and the Cobb angle is simply the angle at the crossing of these two lines. However, The Cobb method is limited because it cannot fully determine the three-dimensional aspect of the spine. It is not as effective, then, in defining spinal rotation. Other diagnostic tools are needed in order to determine a more accurate assessment. One means of evaluation of the rotational aspect of scoliosis is by using an Inclinometer. The Inclinometer measures axial trunk inclination (ATI) in a forward bending position. This measurement is performed while the patient stands with his/her feet together and knees straight and is asked to bend forward at the waist. While bending, the examiner looks for asymmetries of the trunk. If asymmetry is observed, it is quantified by an inclinometer, which indicates the magnitude of the rotational prominence.

The combination of mapping the position of spinal prominences of a patient and measurement of ATI can provide accurate measurements of spinal deformity in a non-invasive manner. Moreover, the combination of the two yields a three dimensional assessment to better understand the deformity. This information is crucial for monitoring and when a clinical decision regarding treatment, such as a surgery, is considered in order to improve the spinal deformity. In these cases the physician has to take into consideration not only the two-dimensional deviation of the spine, as observed by the Cobb angle but also the rotation of the vertebrae. When taking into consideration only one factor, the consequences of the clinical intervention (conservative or surgical) could be a deterioration of the patients body balance. Yet before deciding on an invasive interference, the patients, which in most cases are young in age, have to go through a series of follow up inspections in order to determine the severity, type and, cause of their deformity, and most important: has there been a deterioration in its condition and has skeletal growth has reached maturity. For these follow up inspection, the physician monitors the child every few months using repeated x-rays as it is, currently, the most cost efficient method for diagnosing scoliosis.

However, as much as it is crucial to follow up on scoliotic children, physicians are trying to decreases patients' exposure to x-ray. Researchers have indicated that there is a strong correlation between multiple diagnostic x-rays during childhood and breast cancer mortality. For example: female patients who had an average of 25 x-ray exams have a 70% higher risk of breast cancer than women in the general population. Hence, experts hope that an accurate, noninvasive, diagnostic technique will eventually be developed to replace most of the x-rays used to monitor the progression of scoliosis. There have been several attempts to overcome the need for x-ray follow up of scoliotic patients. One of these methods is the surface topography, which involves the study of the 3D shape of the surface of the back. These methods do not involve ionizing radiation and use direct measurement of the patient's back or surface reconstruction from scanned light or photographic techniques. However, these methods can only provide information on the trunk asymmetry and spine symmetry line and are limited in their application and their direct relation to radiographic measures is uncertain, as no direct information regarding the spine is provided.

Another group of methods used for scoliosis detection and follow up are the Moiré and ISIS topography. These systems produce a true 3D surface representation of a single video photographic image of a fringe pattern projected onto the patient's back. Marker dots are then placed over T1 to T12 (Thoracic vertebrae 1 and 12 respectively) and are observed by a camera, which transfers the data to a computer system to reconstruct the surface representation of the patient. These methods once again only provide information related to trunk asymmetry with out providing information regarding the true spine.

A number of alternative systems are described in the literature for measuring spine curvature in order to avoid the health hazard of radiation; see for example U.S. Pat. Nos. 2,324,672; 4,036,213; 4,600,012; 4,664,130; 4,760,851; 5,251,127; and 5,471,995. However, no system has yet proved to be entirely satisfactory. Efforts are therefore continually being made to develop systems, devices and methods for measuring the spinal curve in a manner which enables more precision, and which can be performed more conveniently, than the existing systems.

In U.S. Pat. No. 6,500,131 Titled CONTOUR MAPPING SYSTEM APPLICABLE AS A SPINE ANALYZER, AND PROBE USEFUL THEREIN, there was disclosed a system and method for imaging the spinal column by detecting the position of the spinal processes of a patient's spinal column, consisting of positioning the patient near a magnetic field generator so that his back is located in that field. Using a magnetic field sensor probe mounted on the examiner's finger, the examiner registers the position of each spinal process, and the data is processed to produce a graphical presentation of the spinal column of the patient. This imaging method involves registering the spinal processes. However, as the orientation the spinal column's vertebras may vary (due to angular rotation—see FIGS. 2a and 2b illustrating two vertebras with a relative angular displacement, causing the spinal processes to appear horizontally shifted with respect to the vertical) recording of the position of the spinal processes alone may not provide enough information, and may produce an image that is slightly distorted with respect to the actual spinal column position.

This system maps the position of the spinal prominences which in the case of thin patients (patients with relatively low body mass) is quite sufficient to determine their spinous processes position. In heavier patients the fat layer cannot be ignored and must be taken into account.

### BRIEF DESCRIPTION OF THE INVENTION

An object of the present invention is to provide an improved method for measuring spinal deformities, using an imaging system such as described in U.S. Pat. No. 6,500,131. This method will provide a more accurate way of mapping the spinal deformity by combining the physiological parameters indicative of position and orientation of the vertebra with the position of spinal prominences of a patient to provide accurate three-plane information of the actual spine in a non-invasive manner.

Yet another object of the present invention is to provide such method that is quantitative.

There is thus provided, in accordance with the present invention, an improved non-invasive method for measuring a spinal deformity of a patient whose spinal prominences representing the spinous processes are registered and their position mapped, the method comprising:
acquiring external physiological parameters indicative of position and orientation of the vertebrae; and
calculating the deformity angle of the spine taking into account the registered spinal prominences and the external physiological parameters.

Furthermore, in accordance with a preferred embodiment of the present invention, the external physiological parameters comprise maximal axial trunk inclination value of the patient.

Furthermore, in accordance with a preferred embodiment of the present invention, the maximal axial trunk inclination is measured in several regions of the spine.

Furthermore, in accordance with a preferred embodiment of the present invention, the maximal axial trunk inclination is recorded and measured using an electromagnetic mapping system.

Furthermore, in accordance with a preferred embodiment of the present invention, the axial trunk inclination is acquired in three different spinal segments: the upper thoracic, the lumbar, and the thoracolumbar segments.

Furthermore, in accordance with a preferred embodiment of the present invention, calculating the deformity angle of the spine is given by SᵢNew=ATI+sᵢ−3 where sᵢ is a deformity angle measured from the registered spinal prominences, and where ATI is the axial trunk inclination.

Furthermore, in accordance with a preferred embodiment of the present invention, calculating the trunk rotation angle of the spine, sᵢ, is given by SᵢNew=BMI*c+sᵢ, where sᵢ describes a lumbar deformity angle and c<10, where c is an axial trunk inclination angle, and BMI is body mass index.

Furthermore, in accordance with a preferred embodiment of the present invention, the method further comprises combining information relating to the external physiological parameters indicative of position and orientation of the vertebrae with the mapped position of the spinal prominences and presenting a three plane view of the spine on a display means.

Furthermore, in accordance with a preferred embodiment of the present invention, the display means comprises a monitor.

### BRIEF DESCRIPTION OF THE DRAWING

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 illustrates the vertebral arrangement of a typically deformed spinal column.

FIG. 2a illustrates a typical vertebra.

FIG. 2b illustrates a horizontally rotated vertebra (with respect to the vertebra of FIG. 2a).

FIG. 3 illustrates a system for imaging the spinal column by registering the position of the spinal prominences of a patient.

FIG. 4 illustrates the use of inclinometer to measure the rotational prominence of a bent-forward patient.

FIG. 5 illustrates a flow chart of an algorithm, in accordance with a preferred embodiment of the present invention, combining the trunk rotation value (ATI) together with position of spinal prominences of a patient to form a 3-plane model of the spine.

FIG. 6 illustrates a flow chart of an algorithm, in accordance with a preferred embodiment of the present invention, correcting the trunk rotation value (ATI).

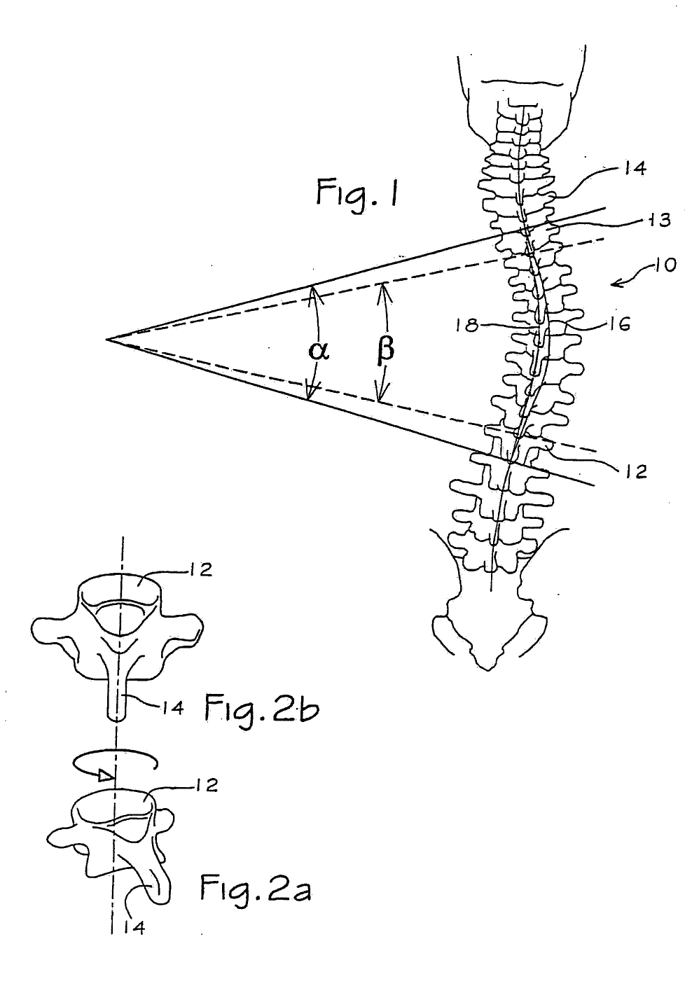

### DETAILED DESCRIPTION OF THE INVENTION AND DRAWING

An aspect of the present invention is the provision of a method that combines contour of the spinal prominences together with the trunk rotation (ATI) value and BMI value to form and display an accurate three-dimensional assessment and graphical image of the spinal deformity. Another aspect of the present invention is a digital method of obtaining the trunk rotation value (ATI) by acquiring data provided by a digital inclinometer.

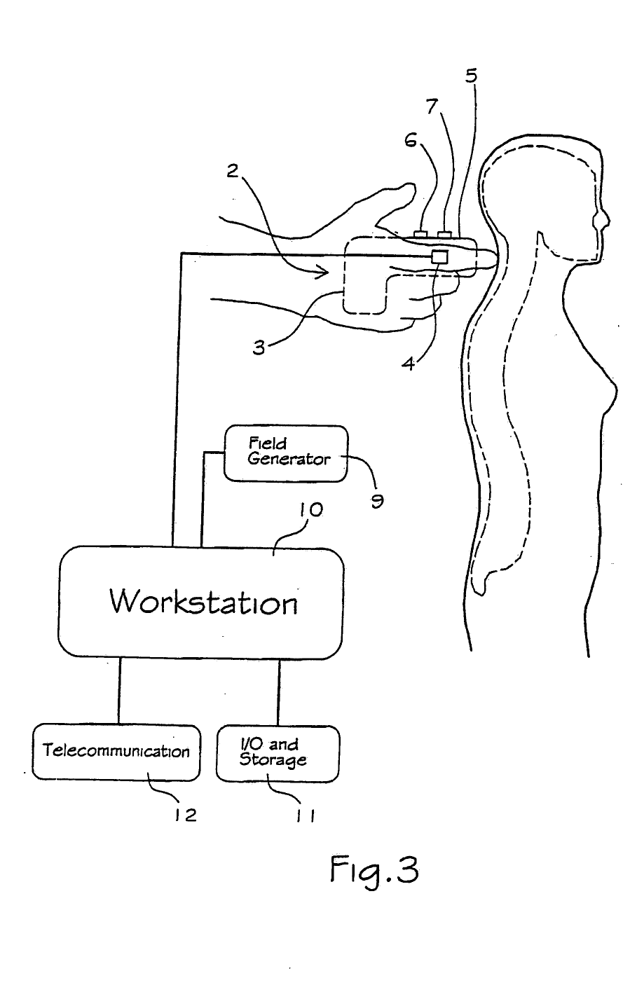

FIG. 1 illustrates the vertebral arrangement of a typically deformed spinal column. The spinal column 10 is made up of stacked vertebrae 12. In a deformed state an angle (Cobb angle) is formed between the inclination of the end-vertebras, which are the vertebrae at the upper 13 and lower 12 limits of the curve, which tilt most severely toward the concavity of the curve. When using the system disclosed in U.S. Pat. No. 6,500,131 (see FIG. 3 illustrating a preferred embodiment of the system disclosed in these patent applications) the arrangement of the spinal vertebrae as imaged is indicated by line 18 adjoining the spinous processes of the spine. However, this line may be distorted with respect to the real line of deformity of the vertebras 16, in case of angular rotation of the vertebras.

FIG. 3 illustrates a system for imaging the spinal column by registering the position of the spinal prominences of a patient. When probe 2 is used, as shown in FIG. 3, for mapping the curvature of a person's spine, the movements of the position sensor 4, which correspond to the curvature of the person's spine, are tracked by a position tracking system included within a data processor in a workstation.

In the preferred embodiment of the invention illustrated in FIG. 3, the position tracking system is of the electromagnetic field type. It includes a transmitter 9 for generating a magnetic field in the space occupied by the person's spine to be mapped. The position sensor 4 within the probe 2 is a tri-axial magnetic sensor for sensing the instantaneous position of the probe within the generated magnetic field. Both the transmitter 9 and the position sensor 4 produce signals which are applied to the workstation, generally designated 10, which tracks the movement of the position sensor 4, and thereby of the probe 2, as the probe is moved with the user's hand along the outer surface of the subject's spine.

The workstation 10 is also provided with a telecommunication channel 12 for communicating with remotely-located medical centers, company servers, on-line technical support, and the like. The workstation 10 further communicates with a storage device and with input-output (I-O) devices (which are denoted by number 11).

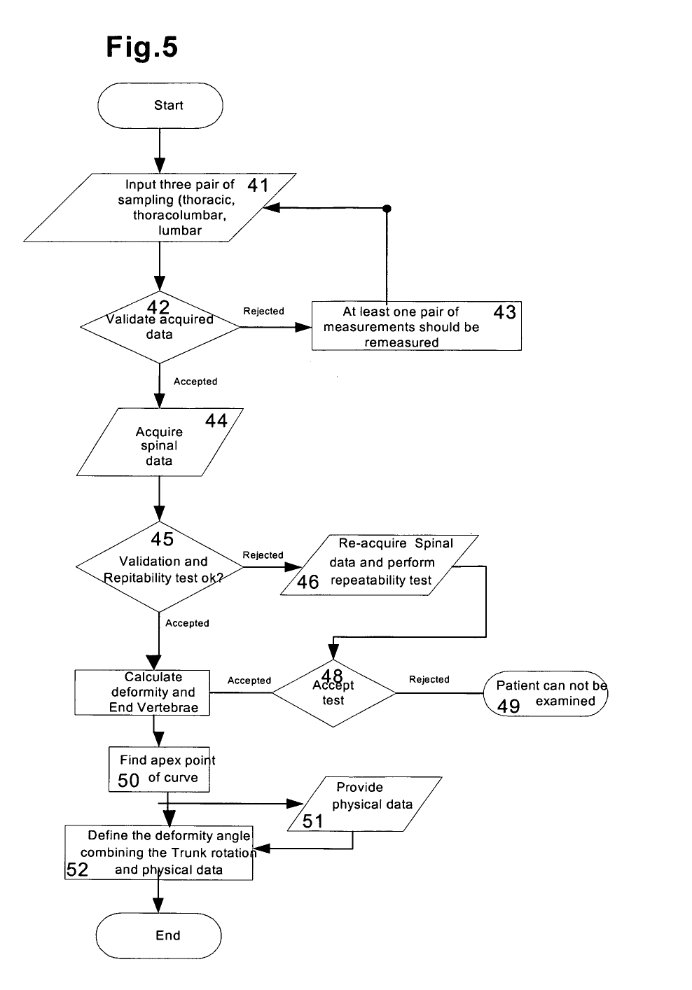

Reference is now made to FIG. 5 describing the flow chart of the algorithm combining the trunk rotation value (ATI) together with the spinous-process deformity angle to form a 3D model of the spine.

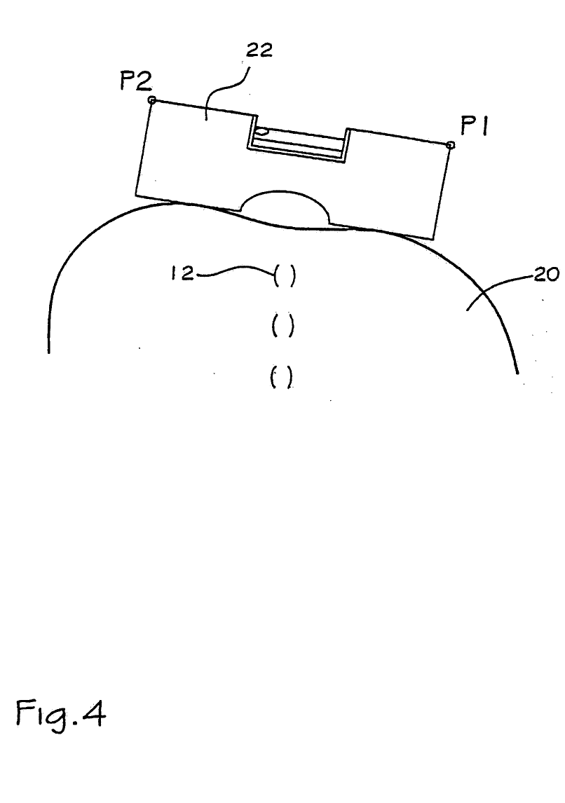

The first steps of the, algorithm marked as steps 41, 42, and 43, relates to data acquisition of the trunk rotation value (ATI). In this stage, the patient is positioned bent over the hip bar with his shoulders at hip level. The user is then requested to provide the system with three pairs of points where each pair is located in a different spinal segment: This data is believed to be essential in order to measure axial trunk rotation angles needed for the algorithm. The angles may be measured using a digital inclinometer or any other type of inclinometer. For each angle, the inclinometer is placed in the relevant spinal segment 12 and then two points are acquired, when the patient 20 is in bended position. The two points are located at the upper left and right part of the inclinometer 22 indicated as P1 and P2 in FIG. 4. The points are acquired using a six degrees of freedom position sensor as described in U.S. Pat. No. 6,500,131. These two points define a line whose angle is essential for the algorithm and are marked as angles a, b, and c.

After acquisition procedure for the three ATI angle is done, the data (angles) is processed in order to verify its validity. In case of one or more of angles, a, b, or, c are off value, the user is asked to repeat those measurements which were out of range. This validation procedure is described in steps 42 and 43 of FIG. 5 and is optional.

In the following steps of the algorithm, described in steps 44, 45, 46, 47, 48, 49, and 50, spine scan is being performed. This procedure is described in U.S. Pat. No. 6,500,131. This part provides, as an output, the following parameters:
sᵢ(i=1, . . . , n), the deformity angle of the spine. n varies usually between 1-3 but can also be more in case of multiple curvature in the spine.

Upper-End-Vertebrae (UEVᵢ)—The most upper vertebrae bounding the curve described by sᵢ.

Lower-End-Vertebrae (LEVᵢ)—The lowest vertebrae bounding the curve described by sᵢ.

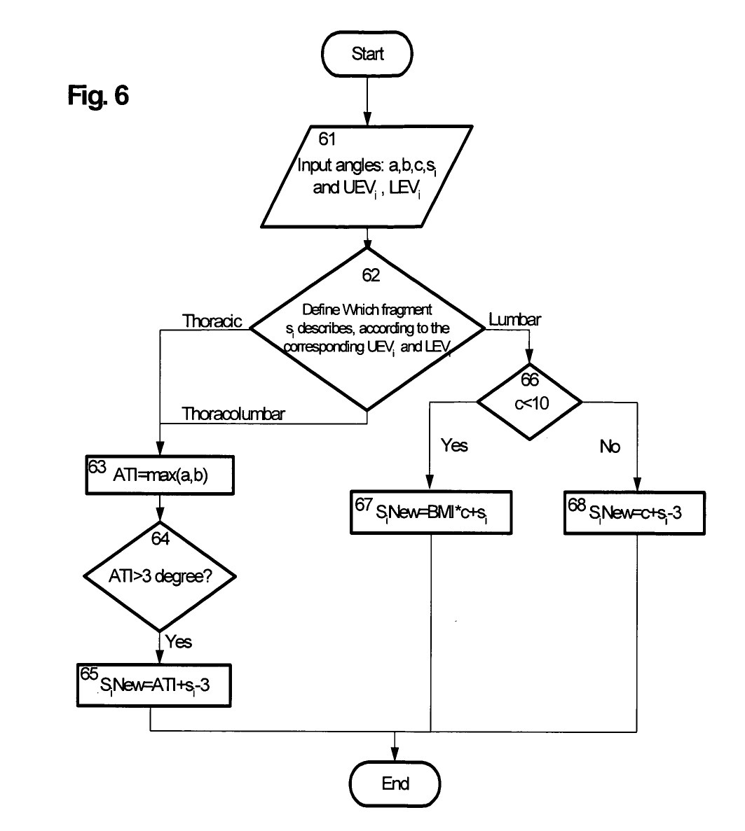

Steps 51 and 52 are the correction steps for correcting the deformity angles, sᵢ, obtained from steps 44 to 50. This correction is essential due to the 3D nature of the spinal curve. Reference is now made to FIG. 6, which describes the algorithm used for data correction presented in steps 51 and 52 of FIG. 5. This algorithm is repeated n times, according to the number of the deformity angles, sᵢ, obtained in the previous section. The algorithm steps are as follows:

Step 61: Input data is obtained from previous calculations. The data includes the following parameters: a, b, and c which are the ATI angles measured by the digitized inclinometer in steps 41 to 43 of FIG. 5, sᵢ which is the ATI angle obtained by steps 44 to 50 of FIG. 5, and UEVᵢ and LEVᵢ obtained by steps 44 to 50 of FIG. 5.

Step 62: Define Which fragment si describes, according to the corresponding UEVᵢ and LEVᵢ.

Step 63: If, according to step 62, sᵢ is a curvature angle of the thoracic or the thoracolumbar spine then define ATI as the higher number between a, and b (i.g if a is bigger then b then ATI equals to a, and visa versa).

Step 64: If, ATI is larger than 3, then correct sᵢ to SᵢNew according to step 65.

Step 66: If, according to step 62, sᵢ is a curvature angle of the lumbar spine then if c is smaller than 10 then correct sᵢ to SᵢNew according to step 67, where BMI is the body mass index. BMI may be calculated as: BMI=Weight*1000/Height² (the Weight of the patient measured in Kg and his Height, measured in cm. For other measurement units a certain factor needs to be added. parameters are obtained in step 51 of FIG. 4). If c is equal or bigger than 10 then correct sᵢ to SᵢNew according to step 68.

All correction equations and constants are the results of a statistical clinical study carried out by the inventors of the present invention.

It should be clear that the description of the embodiments and attached figures set forth in this specification serves only for a better understanding of the invention, without limiting its scope.

It should also be clear that a person skilled in the art, after reading the present specification could make adjustments or amendments to the attached figures and above described embodiments that would still be covered by the scope of the present invention.

1. An improved non-invasive method for measuring a spinal deformity of a patient whose spinal prominences representing the spinous processes are registered and their position mapped, the method comprising:
acquiring external physiological parameters indicative of position and orientation of the vertebrae; and
calculating the deformity angle of the spine taking into account the registered spinal prominences and the external physiological parameters.

2. The method of claim 1, wherein the external physiological parameters comprise maximal axial trunk inclination value of the patient.

3. The method of claim 2, wherein the maximal axial trunk inclination is recorded and measured using an electromagnetic mapping system.

5. The method of claim 2, wherein the axial trunk inclination is acquired in three different spinal segments: the upper thoracic, the lumbar, and the thoracolumbar segments.

6. The method of claim 2 wherein calculating the deformity angle of the spine is given by SᵢNew=ATI+sᵢ−3 where sᵢ is a deformity angle measured from the registered spinal prominences, and where ATI is the axial trunk inclination.

7. The method of claim 2 wherein calculating the trunk rotation angle of the spine, sᵢ, is given by SᵢNew=BMI*c+sᵢ, where sᵢ describes a lumbar deformity angle and c<10, where c is an axial trunk inclination angle, and BMI is body mass index.

8. The method of claim 1 further comprising combining information relating to the external physiological parameters indicative of position and orientation of the vertebrae with the mapped position of the spinal prominences and presenting a three plane view of the spine on a display means.

9. The method of claim 9 wherein the display means comprises a monitor.

10. An improved method for measuring a deformity angle of a spine of a patient substantially as described in the present specification and accompanying drawing.
